(19) United States
(12) Patent Application Publication
(10) Pub. No.: US 2006/0174360 A1
(43) Pub. Date: Aug. 3, 2006

(54) Non-inbred embryonic stem cells having good developmental potential

(76) Inventor: Alan Clark Peterson, Montreal (CA)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/562,952

(22) PCT Filed: Jul. 8, 2004
(86) PCT No.: PCT/CA04/00998

§ 371 Date: Apr. 5, 2006

Related U.S. Application Data
(60) Provisional application No. 60/485,430, filed on Jul. 8, 2003.

(51) Int. Cl. A01K 67/027; C12N 5/06

(52) U.S. Cl. 800/18; 435/354

(57) ABSTRACT

The present invention provides non-inbred mouse embryonic stem (ES) cells comprising alleles derived from at least two different mouse strains and having high developmental potential. The ES cells optionally comprises a docking site for a transgene. Methods for production of the ES cells are also disclosed. This invention further provides chimeric and transgenic mice derived from the non-inbred ES cells and methods for deriving such mice.

### FIELD OF THE INVENTION

The present invention pertains to the production of mice with defined genetic properties, particularly the production of transgenic mice using embryonic stem cells.

### BACKGROUND

In the past, considerable effort has been invested in producing transgenic non-human mammals, such as mice, and during that time, a variety of methods have been developed.

One technique for introducing foreign genetic material into animals makes use of the potential of embryonic stem cells (ES cells) to create chimeric animals. Embryonic stem cells are derived from the inner cell mass (ICM) of blastocysts; they are totipotent cells which are capable of developing into all cell lineages, including germ cells, when introduced into an embryo by injection into diploid blastocysts or by aggregation with morulae (Robertson, (1987) in Teratocarcinomas and Embryonic Stem Cells: A practical approach, ed. Robertson, E. J. (IRL Press, Oxford Washington D.C.), pp. 71-112; Bradley, (1987) in Teratocarcinomas and Embryonic Stem Cells: A practical approach, ed. Robertson, E. J. (IRL Press, Oxford Washington D.C.), 113-151; Beddington and Robertson, (1989) Development 105, 733-737; Nagy et al., (1990) Development 110: 815-821). ES cells can be isolated from blastocysts and then established as permanent cell lines, if they are cultivated under well defined culture conditions which are strictly adhered to, and they can be genetically manipulated. In view of this ability, they constitute an effective tool for modifying the mammalian, and particularly the mouse, genome by means of controlled mutations or other genetic modifications in ES cells that are subsequently transmitted into animals.

In recent years, various experimental techniques have been developed for producing animals derived from totipotent cells, such as ES cells. In the case of ES cells the primary objective of these methods was to maintain the entire developmental potential of ES cells in vitro and to use host cells in the formation of chimeras selected or manipulated to have restricted developmental potential and thus increase the frequency of forming chimeras capable of germline transmission (Nagy, et al. (1990) Development 110: 815-821; Kaufman and Webb, (1990) Development 110, 1121-1132).

In early studies, various ES cell lines were aggregated with morulae in order to produce foetuses which are derived completely from ES cells (organisms derived completely from ES cells are hereinafter referred to as ES animals, e.g. ES mice or ES foetuses); however, the ES foetuses obtained died at birth (Nagy, et al. (1990) Development 110: 815-821). Further studies showed that viable, fertile ES mice derived exclusively from ES cells can be obtained if wild-type R1 cells of an earlier passage (Nagy, et al., (1993) in Gene Targeting: A practical approach, ed, Joyner, A. L. (IRL. Press, Oxford N.Y. Tokyo), pp. 147-180) or TT2 cell lines (Ueda et al., (1995) Jikken-Dobutsu 44, 205-210) are used for the aggregated with tetraploid morulae.

Most mouse ES cell lines are derived from inbred mouse strains. The use of these ES cells in generating cloned mice has, unfortunately, been complicated by the fact that many of the cloned animals suffer from congenital abnormalities and die shortly after birth. Embryonic and fetal losses are also extremely high, such that typically less than 1% of the manipulated embryos will give rise to live born animals. Recently, it has been hypothesized that genetic heterogeneity in the ES cells may contribute to their developmental potential (Cross (2001) Proc. Natl. Acad. Sci. USA 98:5949-5951; Eggan, et al. (2001) Proc. Natl. Acad. Sci. USA 98: 6209-6214) since mouse ES cells derived from F₁ crosses of inbred mouse strains were found to have improved developmental potential, or hybrid vigour, in comparison to ES cells derived from the inbred strains. These F₁ hybrid ES cells have been used in a method for producing ES mice using tetraploid blastocysts (U.S. Patent Application No. 20020078470).

Since its introduction in 1993, (Nagy, et al., Proc. Natl. Acad. Sci. USA 90: pp 8424-8428) the ES cell line designated R1 has enjoyed wide use as a robust cell line in various gene targeting, chimera and cloning experiments. R1 cells were derived from a blastocyst obtained from a cross between two 129 sub-strains, 129/Sv and 129/Sv-CP. The original article by Nagy showed that R1 cells, if obtained from early passage, or from one of five later passage sub-clones (passage 12), also had the ability to produce completely ES cell-derived mice when aggregated to tetraploid embryo. Such mice were found to be both viable and fertile.

Subsequently, a thorough examination of 129 sub-strain relationships was conducted by two laboratories (Simpson, et al., Nat Genet. 1997 May;16(1):19-27 and Threadgill, et al. Mamm. Genome. 1997 June;8(6):390-3). Both studies demonstrated that extensive genetic differences exist between the 129 sub-strains. Notably, the extent of the inter sub-strain differences revealed by genome sequence polymorphisms led to the proposal that the 129/SvJ strain should be described as a recombinant congenic strain (129cX/Sv), being derived from 129/Sv and an unknown strain. Furthermore, extensive genetic variation was found amongst all 129 sub-strains. This variation was apparently introduced by genetic contamination or as residual heterozygosity from various backcrossing programs. This led to a complete revision of nomenclature for strain 129 mice by the Committee on Standardized Genetic Nomenclature for Mice (Festing et al., Mamm Genome. 1999 August;10(8):836).

Thus, the founders of the R1 cell line, 129/Sv and 129/Sv-CP, beyond demonstrating obvious differences in coat appearance (129/Sv mice have a chinchilla coat colour while 129/Sv-CP are agouti), have extensive allelic differences. Taking experimental advantage of this extensive heterozygosity, it was possible to track chromosome segments within R1 ES cells undergoing selection for homozygous targeted gene disruptions (Lefebvre, et al., Nat Genet. 2001 March;27(3):257-8). Because the extent of heterozygosity observed in R1 cells approaches or equals that expected in F₁ cells obtained from matings between the variously related typical inbred strains, the hybrid R1 cells are expected to demonstrate significant heterosis and that feature is assumed to be a major contributing factor to their developmental capacity in chimeras. Notably, their multiply heterozygous constitution (heterosis) would contribute to their demonstrated ability to support the generation of 100% ES cell derived mice when introduced into tetraploid preimplantation embryos to generate chimeras. Similarly, the nuclei of R1 ES cells also were shown to direct development to term of cloned mice (Gao S, et al., Biol Reprod. 2003 February;68(2):595-603) and to maturity (Wakayama T, et al., Proc. Natl. Acad. Sci. USA 1999 Dec. 21;96(26):14984-9).

There remains a need, however, for ES cells having maximal heterosis and related developmental potential. Advantageously, such cells would also have the capability of being easily modified to include a transgene, or combination of transgenes.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide non-inbred (outbred) mouse embryonic stem cells having good developmental potential and optionally containing a transgene docking site. In accordance with an aspect of the present invention, there is provided a non-inbred mouse embryonic stem (ES) cells comprising alleles derived from at least two different inbred mouse strains and optionally comprising a transgene docking site, wherein said stem cells have good developmental potential.

In accordance with another aspect of the present invention, there is provided a non-inbred mouse embryonic stem (ES) cells comprising alleles derived from at least three different inbred mouse strains and optionally comprising a transgene docking site, wherein said stem cells have good developmental potential.

In accordance with another aspect of the invention, there is provided a process for producing an ES cell-derived mouse comprising the steps of introducing non-inbred mouse ES cells into a mouse blastocyst under conditions that result in production of at least one embryo; transferring the resulting embryo(s) into an appropriate foster mother, such as a pseudopregnant female mouse; and maintaining the foster mother under conditions that result in development of live offspring, wherein the ES cells have good developmental potential and optionally comprise a transgene docking site.

In accordance with another aspect of the invention, there is provided a process for producing a transgenic mouse comprising the steps of introducing transfected construct sequences into non-inbred mouse ES cells comprising a transgene docking site and alleles derived from at least two different inbred mouse strains; maintaining the ES cells under conditions that result in homologous recombination at the transgene docking site such that the transfected construct sequences are incorporated in the genome of the ES cells; introducing the resultant recombinant ES cells into blastocyst(s), under conditions that result in production of at least one embryo; transferring the resulting embryo(s) into an appropriate foster mother, such as a pseudopregnant female; and maintaining the foster mother under conditions that result in development of live offspring, wherein the ES cells have good developmental potential.

In accordance with another aspect of the invention, there is provided an ES cell-derived mouse which is derived from non-inbred mouse ES cells comprising alleles derived from at least two different inbred mouse strains and optionally a transgene docking site, wherein said ES cells have good developmental potential.

In accordance with another aspect of the invention, there is provided a transgenic or mutant mouse derived from non-inbred mouse ES cells comprising alleles derived from at least two different inbred mouse strains, wherein said ES cells have good developmental potential.

In accordance with another aspect of the invention, there is provided a transgenic mouse derived from non-inbred mouse ES cells comprising alleles derived from at least two different inbred mouse strains and a transgene docking site, wherein said ES cells have good developmental potential and wherein a transgene has been introduced into the transgene docking site.

### BRIEF DESCRIPTION OF THE FIGURES

FIG. 1A shows a schematic representation of a DNA construct comprising a lacZ reporter gene promoted by hsp68 promoter which constitutes a sensitive enhancer trap. FIG. 1B depicts a lumbar spinal cord and attached roots from a mouse having the above construct as a transgene at the HPRT docking site. This sample was subjected to a sensitive histochemical staining technique to reveal those cells that express the lacZ reporter gene. Except for the large central blood vessel, no other spinal cord or spinal root cells are labelled demonstrating that the HPRT docking site is neutral.

FIG. 2 depicts the pedigrees for BPES cell lines derived according to one embodiment of the present invention.

FIG. 3 is a graphical representation of the proportion of offspring, in first litters, derived from ova fertilized by ES cell-derived sperm and C57Bl/6-derived sperm resulting from a cross of BPES-2 and BPES-5 containing male chimeras with wild type C57Bl/6 females.

FIG. 4 is a graphical representation of the growth in culture of twelve BPES cell lines of the present invention, which demonstrates that all the cells lines have a very similar growth rate.

FIG. 5 is a photograph of transgenic mice produced according to one embodiment of the present invention. These ES transgenic mice are derived from BPES-5 cells containing a myelin basic protein (MBP) regulated transgene injected into a C57Bl/6 blastocyst. Three of the five mice have coats that are dominated by the agouti coat colour and one has a coat that is exclusively agouti which represents 100% ES cell-derived coat colour.

FIG. 6 show FACScan analysis results based on the expression of green fluorescent protein (GFP) from a single copy of an MBP-eGFP transgene in a chimera derived from BK4 cells (as described by Bronson 1994) cells bearing a HPRT docked transgene and wild type C57Bl/6 cells.

FIG. 7 depicts a lumbar spinal cord and attached roots from a chimera derived from a normal C57Bl/6 blastocyst injected with BPES-5 cells bearing a lacZ reporter gene construct regulated by MBP promoter/enhancers. This transgene was expected to express in oligodendrocytes (CNS) and myelin forming Schwann cells (PNS). Dorsal root ganglia contain neurons and satellite cells that do not express the transgene and consequently do not label. In contrast, myelin forming Schwann cells in spinal roots and oligodendrocytes in spinal cord are intensely labelled. The uniform staining of these tissues demonstrates that the transgene-bearing BPES cells dominated in the development of the chimera such that cells of the ES lineage comprise most, if not all, of the Schwann cell and oligodendrocyte population in the chimera.

FIG. 8 depicts a schedule, from DNA transfection to the birth of ES cell dominated chimeras, using a method for producing transgenic mice according to one embodiment of the present invention. The leftmost column lists the different constructs that were transfected and subsequently inserted in the HPRT docking site as transgenes. NFH-1-1 is a construct in which intronic sequences from the mouse NFH locus are added to the hsplacZ sequence represented in FIG. 1. The remaining 4 constructs are based on the same hsplacZ sequence but carry different putative regulatory sequences derived from the mouse myelin basic protein locus.

FIG. 9 is a table that summarises the results of a comparison of transgenic mouse production from BK4 and BPES-5 cells. The results demonstrate that the BPES-5 cells support structure/function analysis with <20% of the effort and expense associated with the use of the BK4 cells and a throughput of 100 s/year. The acceleration supports iteration (new born chimeras: 30 days and newborns in lines: 90 days).

FIG. 10 depicts a typical litter of chimeras derived from BPES cells injected into C57Bl/6 blastocysts. Occasional mice have black coats demonstrating no contribution from the ES lineage. In contrast, however, the majority of chimeras demonstrated a 100% agouti coat and thus appear to be 100% ES cell derived.

FIG. 11 depicts developmental expression of human and chicken Module 4 (Mod4). A: Histochemical staining of spinal cord from transgenic lines. The sequence of Mod4 (FIG. 1) from human and chicken were ligated to hsp/LacZ reporter gene and used in targeted transgenesis. hM4 drives high expression in spinal roots at P11 during myelination and still express in adults. The chicken construct does not give detectable expression during myelination (P11) but expresses specifically in the PNS of adults. B: β-galactosidase activity assay (mU/mg of protein) in sciatic nerves shows that hM4 drives higher levels of expression during myelination (P21) than in adult mice. In contrast the chicken construct, expresses at higher level in adult.

FIG. 12A depicts SA reporter construct expression in Schwann cells throughout development. LacZ expression driven by SA is first observed in E15.5 embryo. Shown here in spinal roots and sciatic nerve, and is maintained during development and in adult mice. FIG. 12B: β-galactosidase activity assay in sciatic nerves shows that SA reporter construct is expressed at low level before myelination (P2) and highly induced during myelination (P10, P21) and lowers in mature mice (3mo). FIG. 12C: Expression of SA construct after sciatic nerve injury. β-galactosidase activity was measured 1, and 2 weeks after nerve crushes in the distal part of injury and compared to expression of the uninjured counterpart.

FIG. 13 shows that different conserved blocks of elements present different functions as assayed by β-galactosidase quantification. Quantification of β-galactosidase activity in sciatic nerves in different lines of mice at P21 or P90. Error bars represents Standard deviation. The P value from a t-test is specified when significant.

### DETAILED DESCRIPTION OF THE INVENTION

The present invention provides multiple non-inbred, or outbred, mouse embryonic stem (ES) cells having improved developmental potential or heterosis and that optionally comprise a transgene docking site. The ES cell lines described herein demonstrate significant heterosis or hybrid vigour, which overcomes many of the practical limitations of the previously available cell lines containing a transgene docking site.

In order to maximise the heterosis and related developmental potential of subsequent ES cell lines, crosses were designed to introduce into blastocysts allelic differences that exist between multiple inbred mouse strains.

In accordance with one embodiment of the present invention, the non-inbred ES cells have alleles derived from two different inbred strains of mice, at least one of which may contain a transgene docking site on an inbred genetic background. Examples of such cells are those designated BPES-1, BPES-2 and BPES-3, which are described in more detail in the Examples. Samples of these cell lines were deposited with the International Depositary Authority of Canada on Jun. 29, 2004 and have been accorded accession numbers 290604-01, 290604-02 and 290604-03, respectively. These ES cells were derived from blastocysts recovered following multiple (5-6) generations of breeding (a combination of crosses and backcrosses from the original mating between the C57Bl/6 female and the BK4 containing chimera (the breeding program was designed to maintain heterosis in mice bearing the HPRT null allele).

Advantageously, the ES cells of the present invention are non-inbred ES cells containing alleles derived from three different inbred strains of mice, at least one of which may contain a transgene docking site on an inbred genetic background. Examples of such cells are designated BPES-4, BPES-5, BPES-6, BPES-7, BPES-8, BPES-9, BPES-10, BPES-11 and BPES-12 and are described in more detail in the Examples. Samples of these cell lines were deposited with the International Depositary Authority of Canada on Jun. 29, 2004 and have been accorded accession numbers 290604-04, 290604-05, 290604-06, 290604-07, 290604-08, 290604-09, 290604-10, 290604-11 and 290604-12, respectively.

In a specific embodiment of the present invention, the first cross was made between a wild type C57Bl/6 female and male chimeras bearing BK4 (HPRT null) and C57Bl/6 cells. The resulting B6129F₁ HPRT+/−females were then backcrossed to wild type C57Bl/6 males or a BC1 male. In this case the breeding program was designed to maintain heterosis in mice bearing the HPRT null allele and introduce further heterozygosity (heterosis) by an outcross to the 129SVPASLCOCRLBk strain of mice.

The non-inbred ES cells of the present invention were derived using a combination of backcrosses, intercrosses and outcrosses to arrive at the blastocysts used for ES cell derivation. This was carried out over many generations as described herein, thereby allowing for natural selection to take place at each generation. This differs from techniques known in the art, which refer to F₁ blastocysts that are derived directly from a cross between two inbred strains.

As used herein, the term non-inbred ES cells encompasses any or all of the herein described non-inbred, or outbred, ES cells and cell lines. Standard techniques, well known to skilled workers, can be used for producing such lines or stocks.

Derivation of the ES cell lines from the non-inbred mouse lines or stocks can be achieved using standard techniques, such as, but not limited to those described in E. J. Robertson (1987) Embryo-derived stem cell lines In Teratocarcinoma and embryonic stem cells: a Practical Approach Ed. by E. J Robertson. IRL Press, Oxford and S. J. Abbondazo, I. Gadi, C. L. Stewart (1993) Derivation of Embryonic Stem cell lines In Methods in Enzymology v. 225 Guide to Techniques in Mouse Development, both of which are incorporated herein by reference. An exemplary method for the production of non-inbred mouse ES cells according to the present invention is described in more detail in Example 1.

The non-inbred mouse ES cells of the present invention demonstrate heterosis, or improved hybrid vigour, in comparison to the ES cells obtained from inbred strains. Although the non-inbred ES cells derived according to the present invention are not identical in terms of pedigree and genetic composition, they all exhibit good developmental potential. The term “good developmental potential,” as used herein, refers to the ability of the ES cells to contribute at a high percentage to the cells of a chimera produced from the ES cells. For certain applications it may be desirable that approximately 100% of the cells of the chimera are ES cell derived, however, 90% or higher is likely a sufficiently high percentage for most applications including in situ or cell based quantitative assays, such as, but not limited to, FACScan. As would be appreciated by a worker skilled in the art, in certain applications, such as establishing the developmental and/or cell specificity expression phenotype of a reporter construct, of the chimeras produced from the ES cells, percentages considerably below 50% are sufficient. Chimeras derived from the ES cells of the present invention typically exhibit greater than 50% ES cell contribution. More commonly the ES cell contribution in the chimera is at or near 100%.

The non-inbred ES cells of the present invention have the ability to compete with normal diploid blastocyst cells to a degree typically associated with ES cells in competition with tetraploid cells (by either aggregation or blastocyst injection). In producing chimeric animals using the non-inbred ES cells of the present invention and wild type C57Bl/6 diploid blastocysts, several chimeras per litter typically are found to be at least 90% ES cell derived and frequently at least one chimera appears to be about 100% ES cell derived. Similar results are obtained in chimeras derived by aggregation.

As used herein, the percentage ES cell contribution, is based on analysis of the chimeras produced using criteria including coat colour, percent germline transmission and distribution of transgene expressing cells within the chimera. It would be readily appreciated by a worker skilled in the relevant art that various methods for determining ES cell contribution may be used. The actual percentage of ES cell contribution observed may vary somewhat depending on the sensitivity of the assay used to identify cells of host cell or ES cell origin.

The ES cells of the present invention may be modified to include a transgene or other genetic modification, which may be introduced using standard techniques. In one embodiment of the present invention the ES cells are modified by targetted disruption of a gene, or genes. Such cells are useful in the production of “knock-out” mice.

In addition to demonstrating good developmental potential, certain of the lines of the non-inbred ES cells of the present invention are designed to include a transgene docking site in order to facilitate their modification to include a transgene or combination of transgenes. The ES cells may contain a combination of a transgene docking site (with or without a docked transgene) and other mutations introduced by recombinant techniques.

A “transgene docking site,” as used herein, is any predetermined site in the genome of a non-human mammal that can be used for single copy introduction of a transgene by homologous recombination or any other form of enzyme-mediated or assisted insertion. The transgene docking site may be a mutated or modified form of an endogenous gene or locus in the genome or it may be an exogenous sequence added to the genome using recombinant techniques. The use of transgene docking sites provide several advantages over random integration of a transgene, which typically results in a wide variation in the level of expression of such heterologous genes among different transformed cells. Further, random integration of heterologous DNA into the genome may disrupt endogenous genes which are necessary for the maturation, differentiation and/or viability of the cells or organism. In transgenic animals derived from random integration of a transgene, gross abnormalities are often caused by the integration of the transgene and gross rearrangements of the transgene and/or endogenous DNA often occur at the insertion site. In contrast, a transgene docking site is used to facilitate single copy insertion of a transgene in such a manner that does not disrupt endogenous genes.

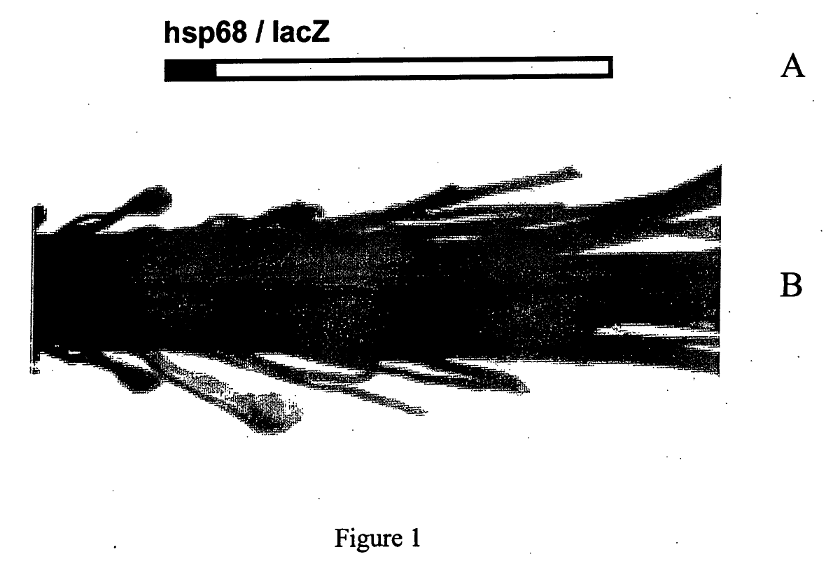

Advantageously, the ES cells of the present invention can comprise a deletion mutant of the X-linked hypoxanthine phosphoribosyltransferase (HPRT) gene as a transgene docking site (S. K. Bronson, et al. (1996) Proc. Natl. Acad. Sci. USA 93:9067-9072). The HPRT deletion mutant is an effective transgene docking site, in part, for the reasons outlined below:
The HPRT docking site is not associated with strong enhancer activity. A neutral reporter gene (lacZ) promoted by the 300 bp “enhancer-trap” hsp 68 promoter derived detectable lacZ expression only in endothelial and cardiac cells of ex-utero mice (FIG. 1).
The HPRT docking site is permissive. For example, it permits detectable expression of a lacZ reporter gene driven by a notably weak MBP promoter sequence (described by Foran and Peterson (1992) J. Neurosci. 12 (12):4890-4897);
The HPRT docking site can reveal different promoter strengths by a difference in the levels of accumulated transgene product (an approximately 25 fold range has been demonstrated) (Farhadi et al, J. Neursci. 2003 and (Denarier, et al. manuscript submitted).
The HPRT docking site faithfully reports construct regulatory functions. In multiple independently derived ES clones (transfection, selection, chimera production, germ-line passage) identical constructs yielded similar qualitative and quantitative results in the subsequent transgenic lines have been demonstrated (unpublished data).

In an alternative embodiment of the present invention the transgenic docking site incorporated in the genome of the ES cells comprises one or more loxP sites. The loxP site is recognised by the site-specific recombinase Cre (Fukushige, et al. (1992) Proc. Natl. Acad. Sci. USA 89: 7905-7909; and Kolb and Siddel (1997) Gene 209:209-216). This site allows the insertion of transgenes that carry flanking loxP sequences. The transgene of interest can be modified to include the flanking loxP sequences using standard methods known to those of skill in recombinant DNA technology.

In a related embodiment of the present invention the docking site the Cre-lox system is used to incorporate into the ES cells a docking site that allows the introduction of the transgene by homologous recombination (Zhao, et al. (2001) BMC Devel. Bio. 1:10). In this case a cassette containing neomycin phosphotransferase (Neo) coding sequence that is expressed from a phosphoglycerate kinase-1 (pgk) promoter flanked by loxP elements is targeted to a chosen locus by homologous recombination. The cells containing the Neo cassette are resistant to the pharmacological inhibitor, G418 and can, therefore, be selected by growth in the presence of G418. G418 sensitivity is restored by removing the Pgk promoter by transient introduction of Cre recombinase, for example, by transiently introducing a plasmid that expresses Cre recombinase. The resulting site is suitable for use as a transgene docking site into which transgenes are introduced by homologous recombination. The transgenes are engineered such that they are upstream of a truncated Neo gene that lacks phosphotransferase activity and can be expressed from a Pgk promoter. This design results in the regeneration of G418 resistance in the cells that contain the transgene.

Another alternative embodiment of the present invention makes use of a φC31 integrase system from Streptomyces phage (Belteki, et al. (2003) Nature 21:321-324) to engineer a transgene docking site.

As would be readily appreciated by a worker skilled in the art, the ES cells of the present invention can comprise any transgene docking site that facilitates site-specific insertion of a transgene into the genome of the ES cells. The selection of the docking site will depend, in part, on the type of transgene to be inserted. Notably, beyond facilitating the controlled insertion of the construct, the chromatin environment and particularly the regulatory influences that emanate from chromatin adjacent to the docking site are relevant to the choice of such site. For example, the ROSA26 locus provides for ubiquitous expression of a reporter gene (Zambrowicz B P et al, Proc Natl Acad Sci USA. 1997 Apr. 15;94(8):3789-94). While neutral sites are advantageous when evaluating the activity of cell specific regulatory sequences, in most cases, permissive sites that allow for transcription of the transgene are desirable.

The non-inbred mouse ES cells of the present invention optionally comprise one or more additional genetic alterations or mutations (e.g. in addition to the incorporation of the transgene docking site). Alternatively, the non-inbred ES cells used can be non-mutant (have not been altered, after they are obtained, to contain a genetic alteration or mutation). The additional genetic alterations or mutations that can be present in the non-inbred ES cells include, but are not limited to, transgenes (cDNA, genes or portions thereof), mutations (targeted or random), conditional mutations, targeted insertions of foreign genes, YAC and BAC sized transgenes, all or part of a chromosome, which may be from the same species as the embryo or another species, such as from a human. They include physical knockout of all or a part of a gene, functional knockout of a gene, introduction of a functional gene and introduction of DNA or a gene portion that changes the function/level of expression of a gene present in the ES cell (e.g., a promoter, enhancer, repressor or a gene portion that encodes antisense or interfering RNA that interferes with post-transcriptional expression). The alterations made in genomic DNA of the non-inbred ES cells can be chosen to produce a phenotype that is similar to (mimics) a condition that occurs in other species (e.g., humans) and the resulting mutant animals (e.g. mice) can, thus, serve as a model for that condition.

Due to the design of crosses used for the production of ES cell lines according to the present invention, such lines will be variously homozygous and heterozygous for alleles that are shared or differ amongst the multiple strains from which they were derived. Following a determination of the distribution of various alleles in a panel of such ES cells, using strain specific genotype markers, this technique can provide opportunities to reveal and evaluate the effects of modifier loci on the expression characteristics or phenotypic consequences of inserted transgenes. Such interactions or “epistasis” is of growing interest in most areas of development and disease research.

Use of the Non-Inbred ES Cells

The invention described herein further relates to a method of producing transgenic mice and to the transgenic mice thus produced. Such mice can be produced with or without the intermediate step of producing chimeric animals, which are subsequently bred to produce the desired transgenic mammal.

In one embodiment, the method of producing an ES cell-derived mouse comprises introducing ES cells of the present invention into mouse blastocysts, under conditions that result in production of an embryo (at least one/one or more embryo) and transferring the resulting embryo(s) into an appropriate foster mother, such as a pseudopregnant female mouse. The foster mother is then maintained under conditions that result in development of live offspring, thereby producing a mouse. The resulting mouse is derived from a single zygote (that which originally gave rise to the ES cells). Such mice are referred to herein as “ES mice” or “ES cell-derived mice”.

Many of the chimeras appear to be at or near 100% ES cell-derived. This conclusion is drawn on observations made: i) in transgene expressing tissues revealed by uniform labelling for a reporter construct; ii) in coat origin revealed by 100% agouti; and iii) in gamete origin revealed by 100% ES derived offspring. Although not all of the chimeras prepared are dominated by the non-inbred ES cell lineage, litters typically contain one or more chimeras that fit this description. Consequently, early access to transgenic mice is made possible without the added difficulties of producing them using developmentally attenuated (e.g. tetraploid) embryos as hosts or partners.

A variety of methods can be used to introduce the non-inbred ES cells into blastocysts, including diploid and tetraploid blastocysts. It has been found that in chimeras derived from the non-inbred ES cells and diploid embryos, the ES cells of the present invention perform in a manner approaching that observed with other cells (e.g. F₁ cells) when used to produce chimeras derived with tetraploid embryos. Therefore, although the ES cells of the present invention can be used with tetraploid partners it is not necessary for successful ES cell chimera production. Rather, the step of generating the tetraploid partners can be avoided altogether.

In one embodiment, the non-inbred mouse ES cells are introduced in blastocyts by injecting the non-inbred ES cells into the blastocysts, such as by microinjection. Alternatively, any other method, which facilitates the introduction of ES cells into blastocysts can be used.

In the case where tetraploid blastocysts are used, the tetraploid blastocysts can be produced using known methods, such as that described by James and co-workers. James, R. M. et al. (1992) Genet. Res. Camb., 60:185. See also Wang, Z-Q et al, Mech. Dev., 62: 137 (1997) and the references cited therein.

In an alternative embodiment of the present invention, the ES cells can be aggregated in vitro with one or more pre-implantation embryos, usually a morula, to produce a chimeric embryo. The embryo is then transferred into a pseudo-pregnant mouse which acts as a foster mother; the chimeric offspring obtained have, in their tissues, different numbers of cells that originate from the original embryo and from the ES cells. Performing this method using the ES cells of the present invention has proven effective in the production of mouse chimeras.

By way of example, BPES-5 clones bearing one of two different HPRT docked lacZ reporter constructs (transgenes) (designated NFHi-4 and 131 bp-5) both gave rise to male chimeras with 100% agouti coat color following aggregation with C57Bl/6 morula and culture to blastocyst followed by transplantation to a pseudopregnant female. This result demonstrates that BPES-5 cells can undergo transfection, homologous recombination at the HPRT locus, subsequent selection in HAT media, co-culture with morula (aggregation) and at least one further day in culture (until blastocyst formation) prior to transplantation and still maintain sufficient developmental potential to dominate in chimeras generated using this non-blastocyst injection method.

The male HPRT wild type ES cell lines BPES-7 and BPES-9 have both given rise to male chimeras (born by Caesarean section on Jun. 14, 2003). with 100% agouti coats. This result demonstrates that non-inbred ES cell lines according to the present invention, without the HPRT deletion, also maintain excellent developmental potential such that they can dominate in the composition of chimeras derived by in vitro aggregation techniques with morula from the C57Bl/6 inbred strain.

In another embodiment, the invention is a method of producing a transgenic mouse by (i) introducing one or more DNA construct sequences into the ES cells, (ii) maintaining the ES cells under conditions that result in homologous recombination at the transgene docking site such that the one or more DNA construct sequences are incorporated in the genome of the ES cells, (iii) introducing the resultant recombinant ES cells into blastocyst(s), under conditions that result in production of an embryo (at least one/one or more embryo) and (iv) transferring the resulting embryo(s) into an appropriate foster mother, such as a pseudopregnant female. The resulting female is maintained under conditions that result in development of live offspring, thereby producing a transgenic ES mouse.

The construct is introduced into the non-inbred ES cells of the present invention using standard transfection techniques. In a specific embodiment of the present invention the transfection is achieved via electroporation. The use of electroporation to introduce DNA into non-inbred mouse ES cells according to the present invention, with specific reference to production of mouse ES cells containing a lacZ reporter gene, is described in more detail in Example 3.

Other methods of transfection exist, including calcium phosphate and various lipid mediated techniques. In addition, a number of viral vectors are known to be effective for introducing a transgene, or combination of transgenes. The actual insertion event (either homologous recombination or random insertion) occurs without further experimental intervention.

Using the non-inbred ES cells according to the invention, transgenic mice can be reproducibly created and these transgenic mice may be used as animal disease models, for studies of gene function, for regulatory genomics studies, for the production of proteins, etc. The non-inbred ES cells and process according to the invention offers the possibility of producing mutant mice rapidly and economically and of having quick access to mutant foetuses and animals, which is a major advantage for research in the field of mammalian biology.

In the case of regulatory genomics, the ES cells of the present invention can be adapted in a controlled fashion to include a putative regulatory sequences in reporter constructs such that different constructs can be compared directly (Denarier, et al. manuscript submitted). The results of one such study are provided below in Example 10.

Unless defined otherwise, all technical and scientific terms used herein have the same meaning as commonly understood by one of ordinary skill in the art to which this invention belongs. All documents referred to in are herein incorporated by reference in their entireties.

To gain a better understanding of the invention described herein, the following examples are set forth. It should be understood that these examples are for illustrative purposes only. Therefore, they should not limit the scope of this invention in any way.

### EXAMPLES

### Example 1

### Procedure for Derivation of ES Cell Lines

Equipment and Materials:

3.5 dpc pregnant mice

Dissecting microscope

M2 & M16 media

Pulled Pasteur pipettes, Gilson P200 tips

Tissue culture plates covered with MEF feeders (4-well, 96-well, 48-well, 24-well, 12-well 6-well, 100 mm)

PBS without Ca and Mg

0.25% trypsin/0.1% EDTA

DMEM with high glucose, with L-glutamine

20% ES cell qualified FBS

100 μM non-essential amino acids

1 mM Sodium Pyruvate

100 μM β-mercaptoethanol

Gentamycin

LIF 2000 U/ml

Procedure

Day 0

Replaced media in feeder wells from 4-well plate with freshly prepared ES media. Flushed 3.5 dpc embryos from uterine horns using M2 medium. Rinsed blastocysts several times through M2. If the embryos were still at morula stage, they were cultured for few hours or overnight in M16 media until they reached blastocyst stage. One blastocyst per well was transferred using a pulled Pasteur pipette, preferably in the centre of the well. A dissecting microscope was placed in the laminar flow hood or next to the Bunsen/EtOH burner on a bench to be used for that and all consecutive procedures in sterile conditions.

Blastocytsts were cultured undisturbed at 37° C., 5% CO₂ for the first 48-72 hours. During that time blastocysts hatched from zona pellucida, attached and formed outgrowths.

Day 3-7

After 48-72 hours, the culture was observed daily to determine the right stage of ICM outgrowth ready for the first disaggregation (usually 5^{th}-7^{th} day after plating). As there was always variability in the size of ICM-derived clump between different embryos, was sometimes necessary to disaggregate these clumps on different days. ICM outgrowth ready for disaggregation should be big enough but not differentiated. 96-well plates with fresh feeders were prepared 24 hours in advance.

Each well was treated separately and no more than 4 wells were done at one time. This was done in order to avoid incubating too long in trypsin.

In the cases where the blastocysts were plated on wells with feeders, the following procedure was performed:
1. Aspirated the media from the 4 wells;
2. Rinsed each well with 0.5 ml of PBS;
3. Prepared a 96-well U-bottom plate by adding 30 μl of 0.25% trypsin/0.1% EDTA solution per well;
4. Using pulled Pasteur pipette with fine capillary, gently circled the ICM clump and removed it from trophoblast cells and placed it into trypsin;
5. Incubated at 37° C. for 10 minutes;
6. Added 30 μl of media to each well to stop trypsin activity;
7. Using a 200 μl capacity pipette, the ICM was broken into smaller clumps of cells (approximately 3-4 cells per clump);
8. Transfered the cell suspension into fresh feeder well of a 96-well plate containing ES media;
9. Repeated the procedure with the remaining embryos;
10. Changed the media after overnight incubation.

Day 8-14

The feeder wells were observed for the production of ES cell-like colonies. The colonies were marked and their growth was monitored day by day. ES cell-like colonies were trypsinized and placed onto fresh feeders according to the following procedure:
1. Aspirated the media from the well;
2. Rinsed with 50 μl of PBS;
3. Added 50 μl of trypsin, incubated at 37° C. for 10 minutes;
4. Added 50 μl of media to each well to stop trypsin activity;
5. The clumps of cells by were broken up by pipetting up and down;
6. Transferred the entire contents of each well into a fresh feeder well of a 48-well plate;
7. Changed the media after overnight incubation;

Day 15-21

The above procedure was repeated as many times as necessary, progressively increasing the size of the well. ES cells formed tight colonies with smooth shiny edges and undistinguishable cells with multiple nuclei. They often formed at the perimeter of the well. When ES cells reached near confluency in a well that was passaged into a fresh feeder well of 6-well plate that was considered “passage 1”. The cells were fed everyday and, when necessary, passaged onto larger plate. Each line was characterized and frozen in cryovials following standard ES cell freezing procedures.

### Example 2

### Derivation of ES Cell Lines

In order to maximise the heterosis and related developmental potential of subsequent ES cell lines, crosses were designed to introduce into blastocysts, allelic differences that exist between multiple inbred mouse strains. Specifically, 129 sub-strains and the C57Bl/6 inbred stain. It has been recognised for several decades that not all combinations of inter-strain alleles are viable accounting for the extensive loss encountered when deriving recombinant inbred (R1) strains. Therefore, in order to select for those combinations of alleles that might promote vs. attenuate developmental potential of ES cells, several generations of breeding were conducted subsequent to deriving blastocysts from which the BPES cell lines were established. By conducting multiple generation crosses, multiple opportunities were introduced for biological selection for viability and fitness at the level of gamete production and fertilizability, subsequent embryo viability, ex utero growth and the subsequent fecundity of the mice derived.

Recognising that:
1) ES cells can be derived with relative ease from “129” strain blastocysts and
2) a general property of early passage ES cell lines is their ability to contribute strongly to chimeras and
3) hybrid cell lines (e.g., R1) demonstrate good developmental potential in the context of blastocyst injection or aggregation chimeras with diploid cells and
4) hybrid cell lines (e.g., R1) demonstrate good developmental potential in the context of blastocyst injection or aggregation chimeras with tetraploid cells and
5) hybrid ES cell lines (e.g., R1) support development in the context of nuclear cloning more robustly than ES cell lines derived from an inbred stain (e.g., 129/Ola).
   ES cells were derived containing a preexisting deletion in the HPRT locus from blastocysts in which extensive heterozygosity had been introduced by breeding. By deriving HPRT deleted ES cells with improved developmental potential, a new level of efficiency was introduced to the controlled strategy of transgenesis reported by Bronson et al. 1996. (Bronson et al. (1996) Proc. Natl. Acad. Sci. USA 93:9067-9072). With such multiply heterozygous cells, both the derivation of chimeras and the establishment of lines bearing HPRT docked transgenes is faster and less expensive.

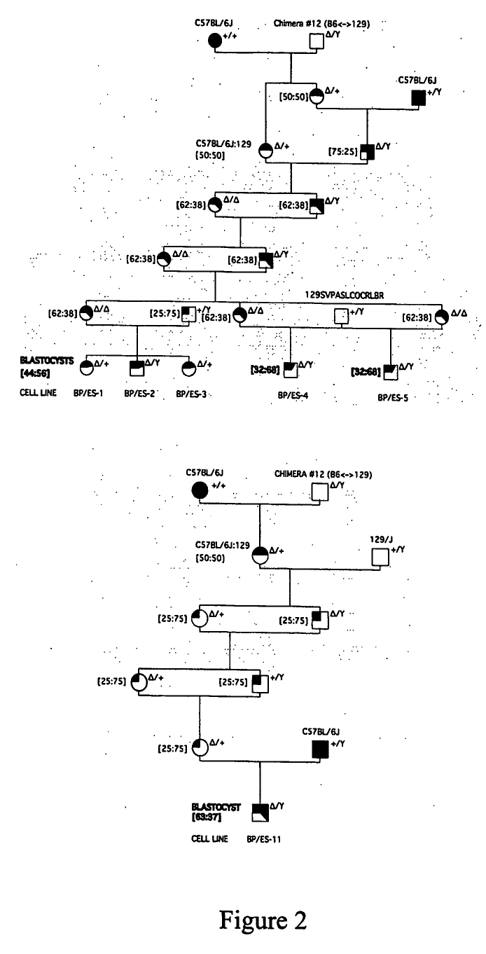

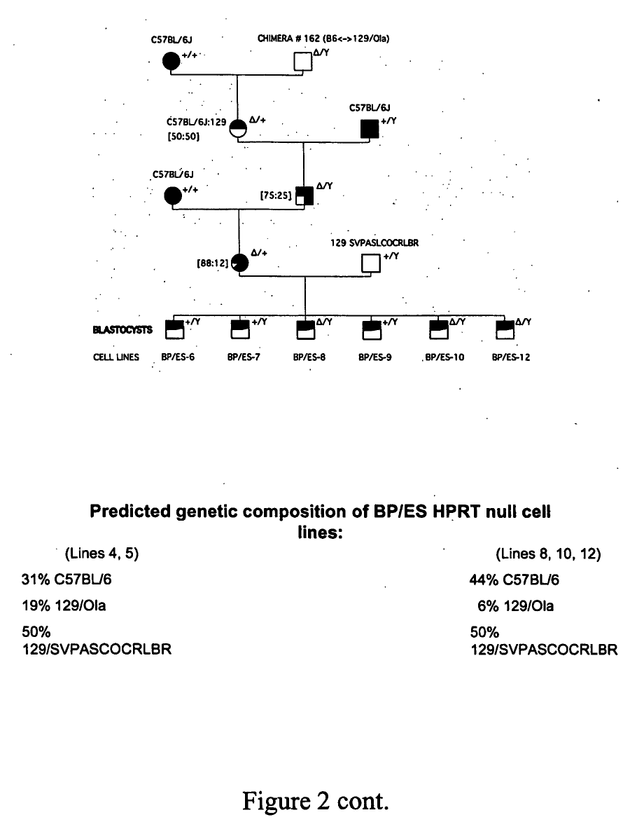

The prexisiting HPRT deleted ES cell line, designated BK4, is a subclone of E14TG2a (Hooper et al., Nature 1987 Mar. 19-25;326(6110):292-5). E14TG2a cells were derived from the 129/Ola sub-strain of 129 mice. BK4 cells were introduced into C57Bl/6 blastocysts by microinjection and male chimeras were derived and mated to C57Bl/6 females. Female offspring were derived from C57Bl/6 ova fertilized by ES lineage sperm. Such B6129F₁ mice were identified by their agouti coat colour and backcrossed to male mice from the C57Bl/6 or 129/J inbred stains. The mating scheme that led to subsequent generations (and incorporating the HPRT deletion) is outlined in FIG. 2.

Five male ES lines bearing the HPRT deletion (designated BPES-4, -5, -8, -10 and -12) were recovered from ova fertilized by sperm from the different 129 sub-line 129SVPASLCOCRLBR. Consequently, these ES cell lines bear unique combinations of alleles from 3 inbred lines (i.e. C57Bl/6, 129/Ola and 129SVPASLCOCRLBR). Subsequent investigation revealed that several of the lines derived in this fashion demonstrate unprecedented developmental potential in the context of chimeras generated either by injection of such cells into wild type C57Bl/6 blastocysts or by aggregation with wild type C57Bl/6 morula. Specifically, amongst the chimeras that develop when such cells are used in standard blastocyst injection procedure, some appear similar in ES lineage composition, to that reported for chimeras derived from F₁ ES cells aggregated with tetraploid embryos or for chimeras derived from F₁ ES cells injected into tetraploid blastocysts, such that amongst the chimeras derived and investigated, the coat colour is frequently dominated or derived exclusively from the ES lineage (ES contribution is agouti vs. black non-agouti from the C57Bl/6 blastocyst). Several such chimeras sire only offspring that are derived from ES lineage sperm. Also, the pattern of transgene expression observed in chimeras bearing transgenes consisting of Tal or a MBP regulated lacZ reporter genes is consistent with the neuronal or oligodendrocyte and Schwann cell lineages (respectively) being close to 100% or completely ES cell derived. Thus, these multiply heterozygous ES cell lines are capable of competing with wild type C57Bl/6 diploid cells with a level of success approaching or equal to that reported for F₁ hybrid cells used in combination with tetraploid embryos (morula or blastocysts). As revealed by some BPES—C57Bl/6 chimeras that demonstrate coat colour chimerism, exclusive or near to 100% ES contribution to chimera tissues is not observed in all chimeras. It should be noted that also in some tetraploid-ES chimeras generated by blastocytst injection, some tissues are found to contain a detectable contribution from the tetraploid blastocyst (Wang, et al. (1997) Mech. Dev. 62:137-145).

The heterosis demonstrated by a number of the BPES cells was achieved using a breeding program involving two or three of the inbred strains 129/Ola, 129SvPAS, 129/J and C57Bl/6J and multiple generations of breeding. One parent of the blastocysts giving rise to male BPES cell lines carrying the HPRT docking site (lines 4, 5, 8, 10 and 12) was a male from the 129PAS inbred strain while the other parents were multiply heterozygous females derived from multiple generations of crossing between C57Bl/6 and 129/Ola and their derivatives as outlined in FIG. 2. Additional BPES cells derived from breeding programs involving the 3 inbred strains are the wild type male lines 6, 7, and 9. The female lines 1 and 3, heterozygous for the HPRT docking site, and the male line # 2 that bears the docking site, were derived from blastocysts recovered from a breeding program involving the 2 inbred strains, 129/OLA and C57Bl/6. Most characterization of developmental potential described here has been done using lines 4 through 10. All evaluation of the HPRT docked transgene bearing lines described above has been done with line 5.

### Example 3

### Characterisation and Use of ES Cell Lines

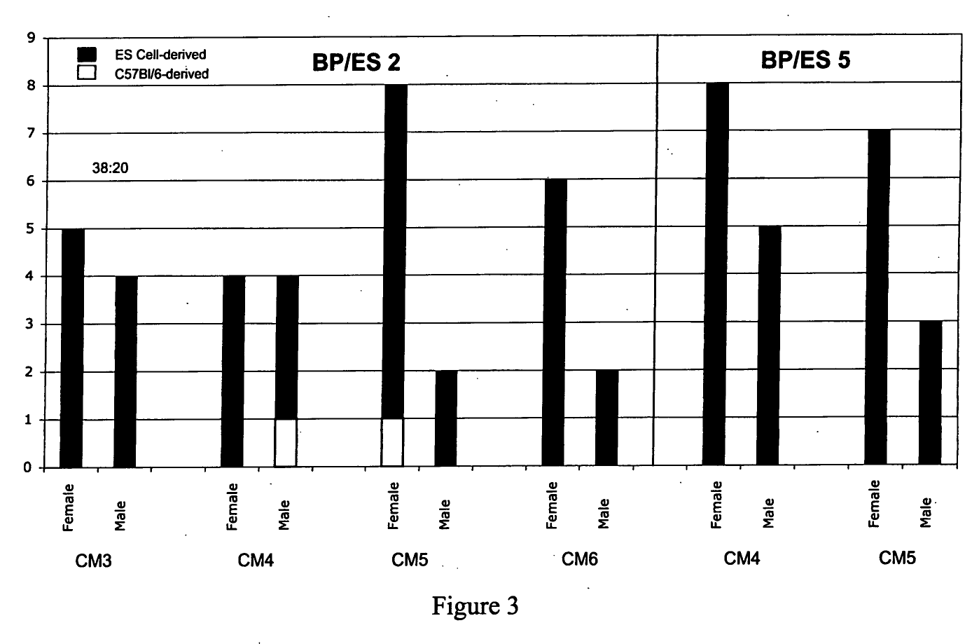

Different ES cell lines according to embodiments of the present invention were derived as described above, each bearing unique combinations of C57Bl/6 and 129 alleles. These cell lines, designated BPES-1 through BPES-12, demonstrated exceptional developmental potential in the context of chimeras produced by injection of ES cells into C57Bl/6 blastocysts. Frequently the resulting chimeras displayed 100% ES derived coat color. Similarly, the germ line in such chimeras appeared to be dominated by the ES lineage; from four BPES-2 and two BPES-5 containing chimeras, 56/58 newborns or fetuses from first mating were derived from ES lineage gametes. (FIG. 3).

To derive these ES cells, stocks containing a deletion of the X-linked HPRT locus were developed on either segregating B6/129 or 129 inbred genetic backgrounds. The HPRT deletion supports a controlled strategy of transgenesis in which a construct is inserted in single copy and known orientation in a precise location in the 5′ to the HPRT coding sequence (Bronson et al. (1996) Proc. Natl. Acad. Sci. USA 93:9067-9072). Multiple breeding strategies were used to achieve blastocysts with a mix of C57Bl/6 and 129 alleles. These breeding strategies are shown in FIG. 2 and described in Example 2.

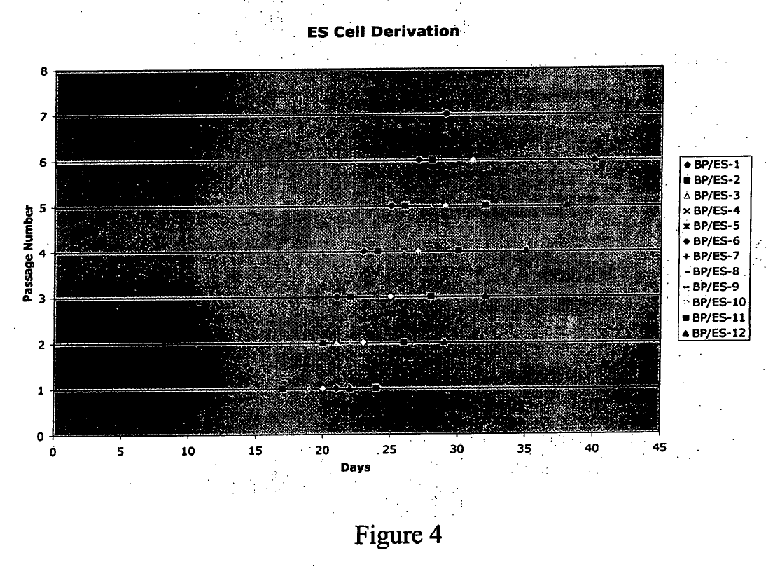

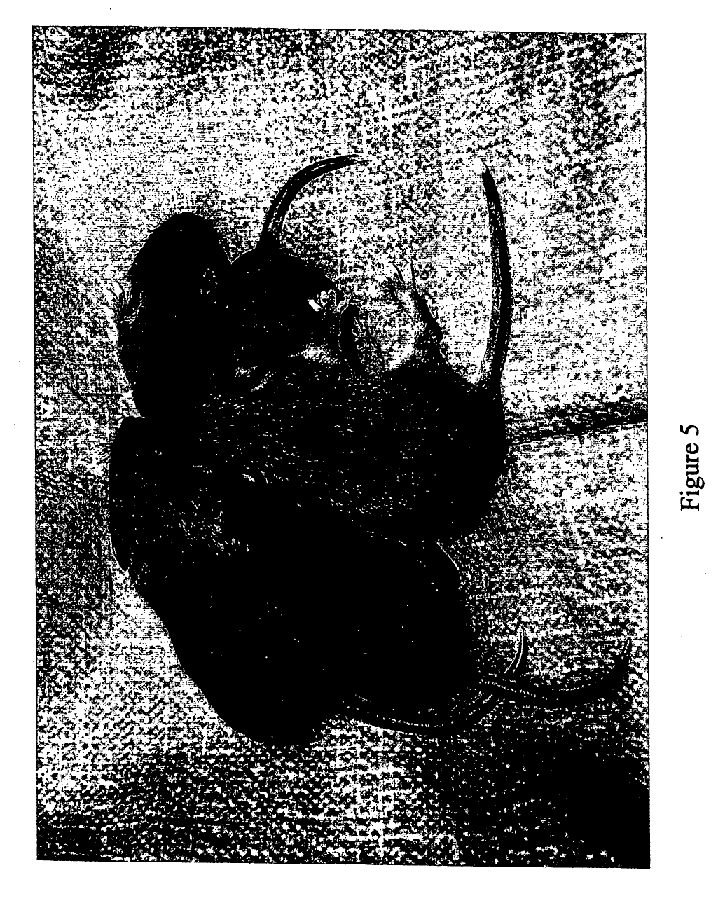

The lines of “mixed” genetic background ES cells all grow well in culture (FIG. 4). The cells are capable of being reliably transfected, maintaining robust developmental potential following freezing/thawing and after clonal selection in hypoxanthine/aminopterin/thymidine (HAT) medium subsequent to restoration of the HPRT locus (via homologous recombination also leading to transgene insertion). (FIG. 5).

The application of these cells in transgenic production appears to offer multiple practical advantages:

1) following a standard transfection protocol, multiple clones typically are recovered. (Standard transfection conditions are: Feed exponentially growing culture of ES cells (˜80% confluent) 2-4 hours before harvesting. Wash the cells twice with PBS. Trypsinize the cells for 8-10 minutes, add media to inhibit the action of trypsin, and pipette the cells up and down to produce a single cell suspension. Centrifuge the cells at 1100 rpm for 5 minutes. Aspirate the supernatant and resuspend the cells in cold PBS at a density of 8×10⁶ cells/ml (this number varies between different labs). Note: the usual yield is ˜3×10⁷ cells per 10 cm dish. Mix 40 μg linear DNA to 0.8 ml of cell suspension into an electroporation cuvette (BioRad, Cat. No. 165-2088). (One 10 cm dish usually allows for 4 different electroporations.) Set the BioRad GenePulser at 240 V, 500 μF. Place cuvette into the electroporation chamber and deliver the pulse. After applying the pulse, place cuvette on ice for 20 minutes. Transfer entire contents of each cuvette onto one 10 cm dish with feeders (some labs prefer to select in the absence of feeders). Media on the feeder dish should be changed to pre-warmed nonselective ES media).

2) their vigorous developmental potential supports an accelerated and enhanced transgenic production schedule.

3) only a small number of chimeras need to be generated in order to have one or more that transmit the ES genotype by fertilizing ova with ES derived sperm.

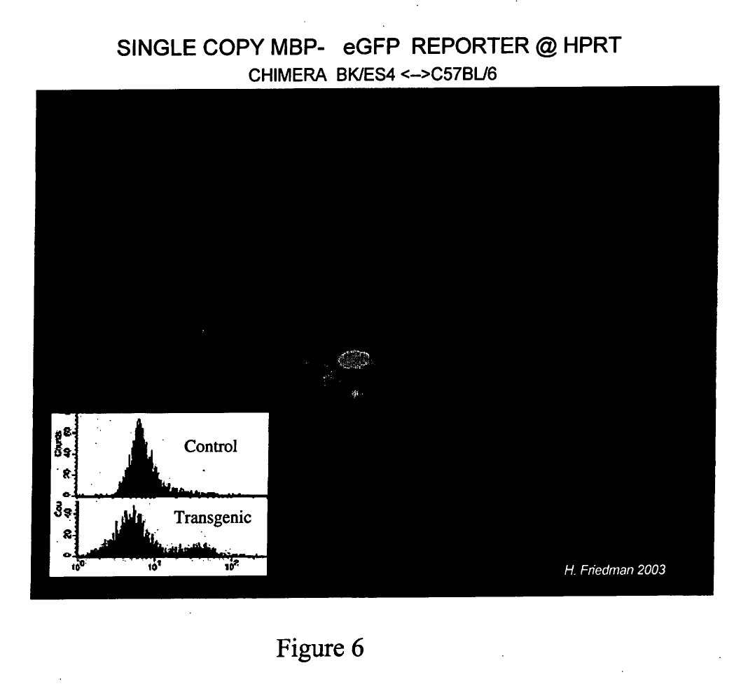

4) ES cell dominance in chimeras supports meaningful qualitative studies (e.g., lacZ reporter gene analysis) and quantitative analysis (e.g., FACScan analysis based upon fluorescent reporter gene expression) directly from chimera tissue (FIG. 6).

Characterisation of Transgene Expression

A 9.4 kb MBP 5′ flanking sequence was ligated to an enhanced green fluorescent reporter gene and introduced into a HPRT targeting vector and BK4 ES cells were transfected. Clones resistant to HAT selection media were recovered and injected into C57Bl/6 blastocysts to generate chimeras. Such chimeras were transplanted to pseudopregnant females and subsequently raised to maturity.

A chimera was sacrificed by lethal injection of the anaesthetic “avertin” and perfused transcardially with a balanced salt solution. Subsequently, the nervous system was recovered and the brain was subjected to tissue disruption and Percoll gradient centrifugation to recover an oligodendrocyte enriched cell suspension. (procedure described in Afshari et al. J. Neurosci Res. 2002 Jan. 15;67(2):174-84). This suspension was analysed using a FACScan apparatus that detects and records the light emission from the eGFP protein. When compared to a similarly prepared cell suspension from a wild type mouse, there is a broad extension of the signal to the right indicating a specific signal is recorded from the eGFP expressing cells in the chimera. From the same chimera, the spinal cord was mounted on a glass slide and viewed with a fluorescent microscope capable of detecting the emission from eGFP. Clearly labelled profiles typical of myelin maintaining oligodendrocytes were observed. It should be noted that this chimera was made with the BK4 ES cell line described by Bronson et al. 1996 (injected into C57Bl/6 blastocysts) and the contribution of the ES cell lineage to the coat (judged by coat color) and the oligodendrocyte lineage (judged by the infrequency of encountering an eGFP expressing oligodendrocyte) and the germ line (as judged by the failure of the chimera to transmit an ES derived sperm) was low.

Transgene Production Schedule

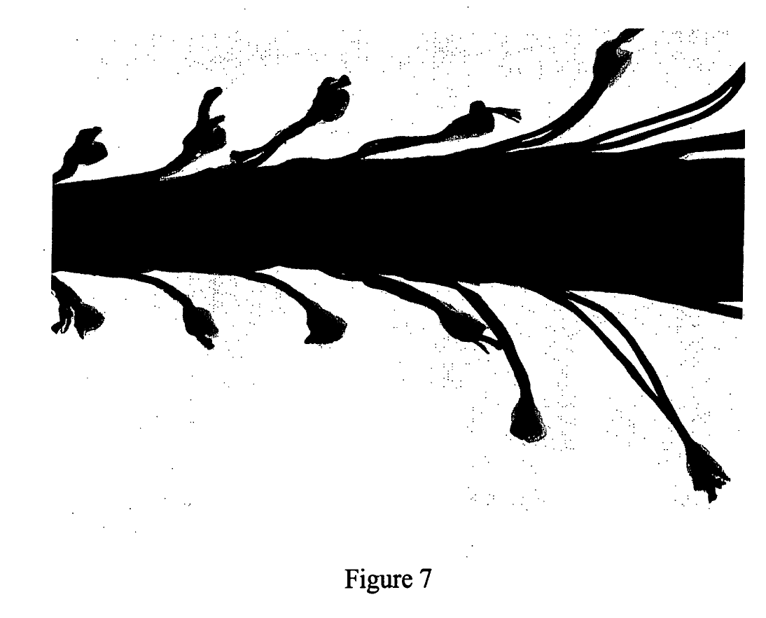

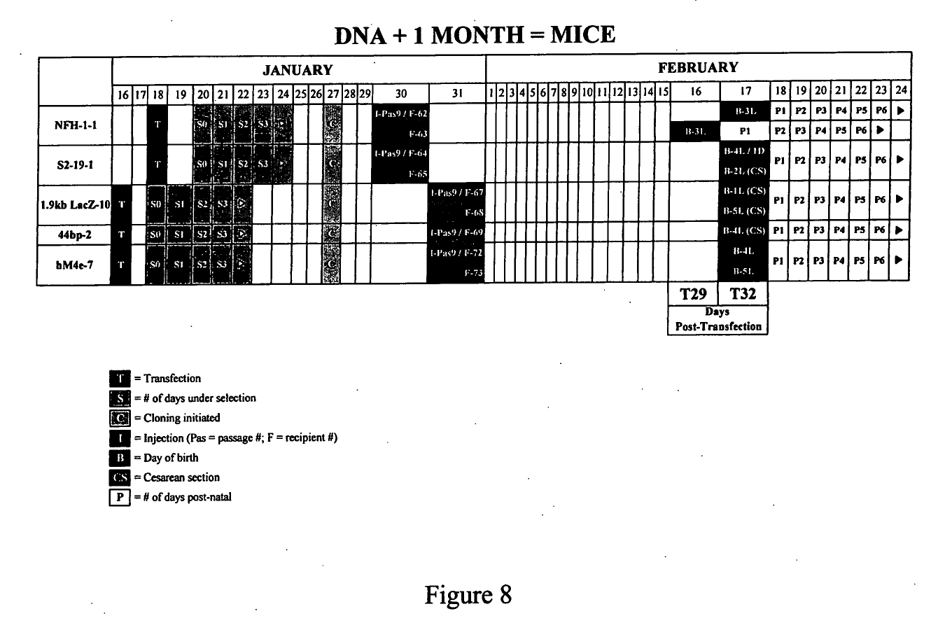

An overall proof of principle experiment was conducted January-March 2003 in which a schedule from DNA transfection to the birth of ES cell dominated chimeras was achieved in 32 days or less (FIG. 7). BPES cells of line 5 were transfected with one of the HPRT targeting constructs as designated in the figure and described above. Following one day in normal culture media, the cells were subjected to selection for those in which the HPRT locus had been repaired using HAT media. Nine or eleven days following transfection clones were recovered and expanded and 12 or 14 days following transfection, blastocysts were injected with cells from the recovered clones to generate chimeric embryos that were transplanted to pseudopregnant females to complete in utero development. New born offspring dominated by transgene bearing ES cells were recovered between 29 and 32 days following the transfection of the DNA HPRT targeting constructs.

### Example 4

### Re-Targeting of Transgene

The wild-type HPRT locus in BPES-6 cells was successfully re-targeted, which lead to the recovery of one clone after 6 thio-guanine (6 T-G) selection against HPRT. Mice born from C57Bl/6 blastocysts injected with derivatives of this clone were born on May 26, 2003. Of 11 live born, 8 were black indicating an exclusive C57Bl/6 origin. The remaining 3 were chimeras: one was a female with a coat 50% agouti; one was a male with a coat 95% agouti and one was a male with a coat 100% agouti. This result demonstrates that the male BPES-6 cells that contain a wild type HPRT allele can be selected using 6 T-G containing media for the loss of HPRT enzymatic activity and such a surviving clone maintains robust developmental potential in chimeras derived from ES injection into C57Bl/6 blastocysts.

### Example 5

### Morula Aggregation

It is widely assumed that injection of ES cells into blastocysts is a more effective technique than aggregation of ES cells with morula as a means of introducing ES cells into chimera such that they dominate in the subsequent embryo and postnatal chimera. (See application of tetraploid blastocyst injections vs. aggregation with tetraploid morula to derive 100% ES cell chimeras). Therefore, in the past, generation of 100% ES cell-derived mice has often relied on the blastocyst injection technique since, in certain circumstances, deriving such mice was not achievable using aggregation techniques.

BPES-5 clones bearing one of two different HPRT docked lacZ reporter constructs (designated NFHi-4 and 131 bp-5) both gave rise to male chimeras with 100% agouti coat colour following aggregation with C57Bl/6 morula and culture to blastocyst followed by transplantation. This result demonstrates that BPES-5 cells can undergo transfection, homologous recombination at the HPRT locus, subsequent selection in HAT media, co-culture with morula (aggregation) and at least one further day in culture (until blastocyst formation) prior to transplantation and still maintain sufficient developmental potential to dominate in chimeras generated using this non-blastocyst injection method.

The male HPRT wild type ES cell lines BPES-7 and BPES-9 also both gave rise to male chimeras (born by Cesarean section on Jun. 14, 2003) with 100% agouti coats using the aggregation technique described above. This result demonstrates that multiple BPES cell lines maintain excellent developmental potential such that they can dominate in the composition of chimeras derived by in vitro aggregation techniques with morula from the C57Bl/6 inbred strain and that the HPRT deletion (transgene docking site) is not required for this developmental potential).

### Example 6

### Transgene Expression

The pattern of expression of MBP and ta1 promoted lacZ reporter constructs was analysed in chimeras with 100% agouti coats. The distribution of transgene expressing cells is consistent with the oligodendrocyte, Schwann cell and neuronal populations of such mice also being derived primarily (more likely exclusively) from the ES cell lineage. This is consistent with the ES origin of the coats and germ-lines in similar chimera.

Histochemical detection of β-galactosidase activity in chimeras bearing ES cells with HPRT docked lacZ reporter constructs. Histochemical staining was performed as described previously (Forghani et al (2001) J. Neuroscience). Briefly, mice were anesthetized and perfused transcardially with 0.5% paraformaldehyde, 2.5% glutaraldehyde in 0.1 M phosphate buffer (pH 7.4). Following post-fixation for an additional hour, whole mounts or tissue sections were incubated at 37° C. in staining solution containing 5 mm potassium ferricyanide, 5 mm potassium ferrocyanide, 2 mm magnesium chloride, 0.02% Nonidet P-40, and 0.4 mg/ml Bluo-Gal Ssigma). Stained whole mount specimens and tissue sections were viewed on a wild M5A stereomicroscope.

### Example 7

### Comparison Study

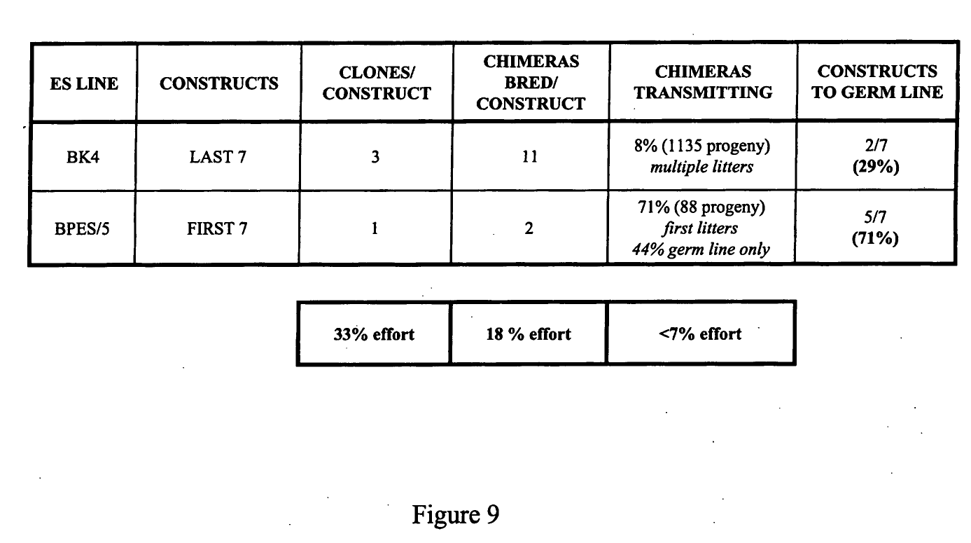

The table shown in FIG. 9 summarises the results of studies in which it was attempted to place 7 constructs into transgenic lines using the BK 4 cell line and 7 constructs into transgenic lines using BPES-5 cells.

In the case of the BK4 cells, in an effort to maximise the chances of obtaining germ-line passage, on average three independent HAT resistant BK4 clones were used per construct to generate chimeras. In contrast, when using the BPES-5 cells only one construct-bearing clone was used per construct. Also, as many BK4 derived chimeras were bred as possible, also in an attempt to maximise the chances of obtaining germ-line passage. In contrast, it was recognised that one good chimera derived from the BPES cells (based upon coat colour) was typically sufficient to pass on the transgene to offspring (and usually in the first litter). In addition, 44% of the chimeras mated (not all selected to be from the 100% ES derived category) gave first litters that were derived entirely from sperm coming from the ES lineage.

A rough calculation of the relative effort (time and expense) is indicated below the comparison of the two types of cells. What this does not reflect are the additional advantages provided by the ES cells of the present invention such as rapid access to analysis of the chimeras containing only, or predominantly, ES cells and the reduction in time required to go from DNA to a transgenic line.

### Example 8

### Chimera Production

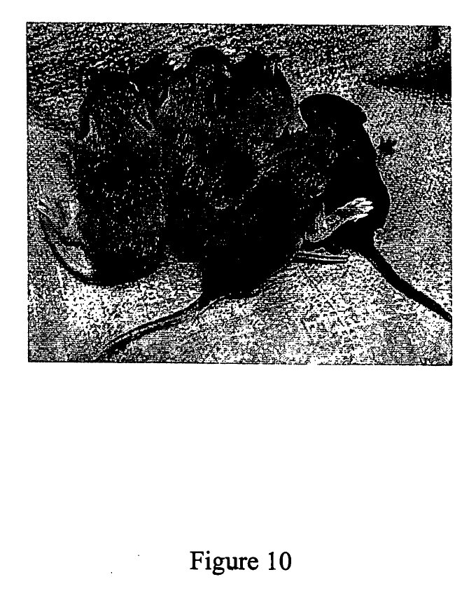

131 different clones have been delivered into 477 chimeras. Demonstrating the high ES cell lineage contribution, chimeras typically had 100% agouti coats (FIG. 10), reporter constructs expressed in the majority of the targeted cell population (See, for example, FIG. 6 of Farhadi et al. (2003) J. Neuroscience 23(32):10214-10223), germ-line transmission occurred in 89.4% of first litters and the sex ratio of chimeras predicts gender conversion in 60% of transplanted female blastocysts.

This high ES cell lineage contribution from the ES cells of the present invention has also been observed in production work reported by Nucleis, SA, a commercial transgenic service provider using BPES cells as described herein.

### Example 9

### Homologous Recombination at Loci Other than HPRT

BPES-6 cells, lacking the HPRT deletion, were used to target a regulatory module (Mod3) in the 5′ flanking sequence of the myelin basic protein (MBP) locus. The Mod3 targeting vector had a 4.6 Kb right arm and 3.7 Kb left arm of homology.

Structure: Left Arm, LoxP-Module-LoxP, pgkNeoLoxP, Right Arm, pgkTK.

The vector was linearized for electroporation and 40 μg (0.45 μg/μl) of DNA was electroporated using 240V; 500 μF; 4 mm cuvette and 8×10⁶ cells. Selection began on day 2 post transfection using G418 250 μg/ml+Gancyclovir 2×10⁻⁶M. Thirty-five clones were recovered on day 8 of selection.

Based on diagnostic Southern blot analysis, three clones were positive for the anticipated targeting event. Chimeras bearing targeted ES clones have been derived.

This study demonstrates that ES cells of the present invention can support homologous recombination at an endogenous site, in this case, the MBP 5′ flanking sequence.

### Example 10

### Application of ES Cells

Methods:

Generation of constructs for HPRT docked transgenesis: The MBP −9.5 kb and −9.0 kb are described in Farhadi 2003. The construct −9.08 kb has been produced by adding a 80 bp PCR product upstream SacII sites of clone −8.9 kb (see FIG. 1B). To clone Mod4 sub-fragments we first generated an hsp-LacZ XS Entry vector, where hsp-LacZ is cloned in Eco RV site of pEntry1a vector (Stratagene). SCE1 sub-domains were ligated upstream of the hsp promoter in reverse orientation according to endogenous MBP. Entry vector are used for in vitro recombination in HPRT Destination vector that include homology arms for HPRT locus using LR recombination reaction kit (Stratagene). The final Destination vector is amplified, sequenced across the insert and linearized by AgeI, 25 to 40 ug are used to transfect ES cells.

Derivation of transgenic mice: Transgenic animals bearing constructs in the HPRT docking site were generated by transfection of Destination constructs bearing the HPRT targeting cassette into BPES-5 cells. Homologous recombination simultaneously restores the deleted HPRT locus in cells and inserts a single copy of the reporter construct into the HPRT 5′ flanking region. Restoration of HPRT expression confers resistance to HAT selection (hypoxanthine, aminopterin, thymidine; (Bronson et al. (1996) Proc Natl Acad Sci USA 93: 9067-72), permitting positive selection for clones derived from the desired homologous recombination event. Following selection and PCR screening, cells were implanted into E3 C57Bl/6 blastocysts, which were then transplanted into the uterus of E2 B6C3F1 mice rendered pseudopregnant by mating with vasectomized males. Chimeras were delivered naturally 16 d later.

Histochemical detection of β-galactosidase activity. Histochemical staining was performed as described previously (Forghani et al. (2001) J Neurosci 21: 3780-7). Briefly, mice were anesthetized and perfused transcardially with 0.5% paraformaldehyde and 2.5% glutaraldehyde in 0.1 M phosphate buffer, pH 7.4. After postfixation for 1 additional hour, whole mounts of dissected organs or tissues sections were incubated at 37° C. in staining solution containing 5 mM potassium ferricyanide, 5 mM potassium ferrocyanide, 2 mM magnesium chloride, 0.02% Nonidet P-40, and 0.4 mg/ml Bluo-Gal (Sigma) or 0.8 mg/ml of X-Gal for tissue sections.

Quantitation of β-galactosidase activity. Sciatic nerves were dissected from male mice and snap frozen in liquid nitrogen. 48 Samples are homogenized using mixer mill apparatus (Qiagen) with 2 minute burst in 250 ul of lysis buffer. Total protein concentrations were then measured for all extracts in triplicate by the Bradford procedure (Bio-Rad) using a BSA standard curve. β-galactosidase activity was detected using the Galacto-Star chemiluminescent assay system (Applied Biosystems) according to manufacturer instructions with readings performed on a Revelation MLX luminometer (Dynex Technologies, Chantilly, Va.). Standard curves were generated with serial dilutions of β-galactosidase (Roche Diagnostics, Hertfordshire, UK) in duplicate.

Results:

Inter-Species Sequence Comparisons Reveal Multiple Conserved Elements within Mod4

The MBP 5′ flanking sequence contains four widely spaced modules of human/mouse conservation (>100 bp at 75% identity) (Farhadi et al. (2003) J Neurosci 23: 10214-23). Module 4, which possesses Schwann cell enhancer activity, lies 9 kb upstream of the ATG in the mouse MBP gene. For this investigation, Mod4 comparisons were extended to rat and chicken sequences and within the 400 bp of conserved Mod4 sequence, the percent identity observed between mouse/rat, mouse/human and mouse/chicken is respectively 91%, 76% and 56%. For the 3 mammalian species, 22 invariant motifs (M) of at least 6 bp were encountered (M1 to M22 FIG. 1B). These total 213 bp. When this comparison was extended to the chicken, 7 invariant motifs totaling 54 bp are identified within the core 241 bp of conserved mammalian sequence.

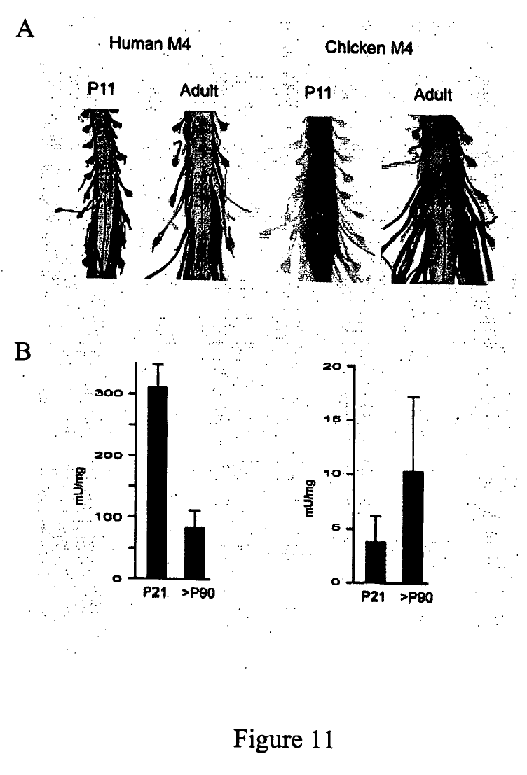

To determine if the Mod 4 sequences from human and chicken could be recognized productively by mouse transcription factors, such Mod4 sequences were ligated to a hsp68 minimal promoter and a LacZ reporter gene. These constructs were docked, in single copy and in a predetermined orientation, in the 5′ flanking sequence of the HPRT locus (Bronson et al. 1996; Farhadi et al. 2003). Despite their diversity, Mod4 sequences from both species drove reporter gene expression in Schwann cells (FIG. 11). The chicken Mod4 reporter was silent during myelin elaboration in pre-weaning animals when both the endogenous MBP locus and human and mouse Mod4 reporters are highly expressed (FIG. 11).

Developmental Regulation and the SA Enhancer

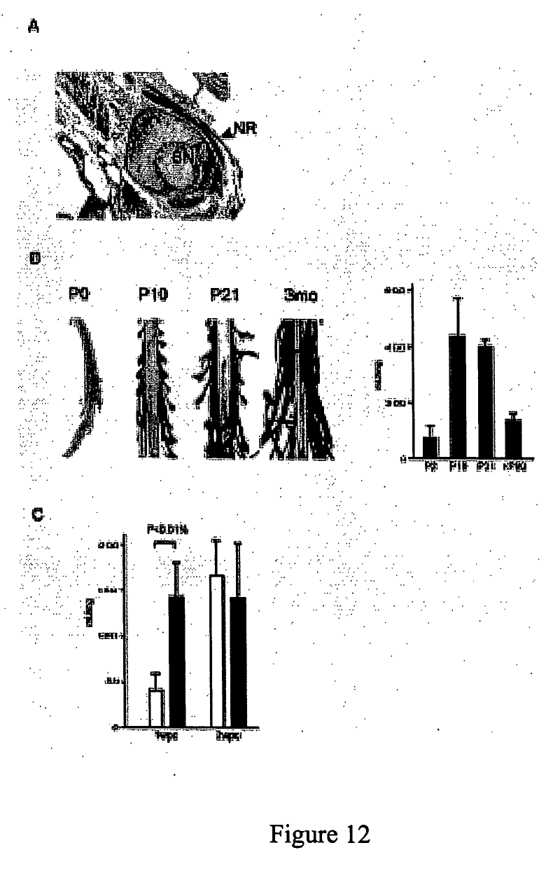

To investigate the phenotype conferred through a 213 bp sequence of the Mod4 (designated “SA”), which contains motifs M10 to M22, in developing mice, fetuses at E15.5 and E18.5 were analyzed for β-galactosidase activity. Sciatic nerves and spinal roots labeled robustly in fetuses of both ages (FIG. 12A). However, during fetal development, individual Schwann cells are associated with multiple axons, myelin elaboration has not begun and MBP up-regulation has not occurred. Thus, the SA regulated reporter construct demonstrated a precocious developmental program suggesting that regulatory sequences located outside SA limit MBP expression in the early stages of Schwann cell maturation.

To evaluate postnatal expression of the SA regulated reporter construct, whole mount preparations were obtained from mice at P0, P10, P21, 3 months, and 1 year of age. Expression was clearly detectable at all ages with staining intensity increasing significantly during the period in which myelin is elaborated (P0 to P10) (FIG. 12B). Quantitative analysis of β-galactosidase activity in sciatic nerve extracts revealed a six fold accumulation of β-galactosidase activity from P2 through P10 followed by a decrease to one third peak activity level by 3 months of age (FIG. 12B).

Regulation of SA Transgene Expression after Transient Loss of Axonal Contact

Following a sciatic nerve crush lesion, axon segments and myelin sheaths distal to the crush site disintegrate in a process called Wallerian degeneration. Schwann cells in the distal stump dissociate from their previously elaborated myelin sheaths, down-regulate expression of myelin genes and dedifferentiate. When axonal contact is re-established with axons ingrowing from the proximal stump, Schwann cells recapitulate features of primary development by ensheathing and remyelinating the newly emerging axons (Gupta et al. (1988) Brain Res 464: 133-41; Trapp et al. (1988) J Neurosci 8: 3515-21; Lamperth et al. (1990) J Neurocytol 19: 756-64; LeBlanc and Poduslo (1990). J Neurosci Res 26: 317-26; Stahl et al. (1990) Brain Res Mol Brain Res 8: 209-12). This experimental paradigm was applied to interrogate SA function in remyelinating nerves. Distal and proximal segments of sciatic nerves in mice bearing the SA construct were analyzed for β-galactosidase activity at 1, 2 and 4 weeks following injury (FIG. 12C). At one week, β-galactosidase activity dropped to ⅓ the level expressed by uninjured contralateral nerves. Two weeks post injury, Schwann cells in the segment distal to the crush have reestablish axonal contact and up-regulated expression was observed. Four weeks post injury, when regeneration and remyelination is largely complete, the expression level in distal segments was indistinguishable from uninjured nerves (FIG. 12C).

Quantitative Phenotypes Conferred Through Mod4 Sub-Domains.

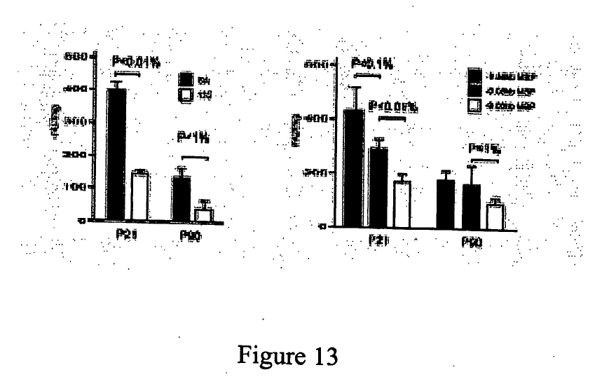

To determine if Mod4 elements not required for Schwann cell targeting modulate expression levels, β-galactosidase activity was measured in the sciatic nerves of mice bearing different Mod4 reporter constructs. The expression level phenotypes realized from the SA and 135 bp constructs was evaluated first. Both sequences confer Schwann cell expression in young and mature mice with the highest levels of β-galactosidase accumulation observed in young animals when active myelin formation has commenced and MBP expression peaks (FIG. 13A). For both constructs, activity measured in preweaning mice was 3 fold higher than in adult mice. Thus, binding sites for the factors distinguishing active myelination from myelin maintenance are present in both sequences regardless of the qualitative or quantitative nature of the underlying transcription factor differences. However, the SA construct is 3 fold more active than 135 bp sequence.

The regulatory activity of the Mod4 sequence located 5′ of Mod4 that contains 9 of the 22 mammalian invariant motifs was valuated next. Three different constructs, all bearing contiguous MBP 5′ flanking sequences but terminating at −9.5 kb, −9.08 kb and −9.0 kb were analyzed. A marked increase in Schwann cells expression level was observed when Mod4 sequences were extended from −9.0 kb to −9.08 kb (FIG. 13A) suggesting that important quantitative information is conferred through one or all of the elements located in this 80 bps. While the expression levels conferred through the −9.08 kb and −9.4 kb sequences were similar in mature mice, significantly lower levels were conferred by the 9.08 kb sequence during myelin formation.

The study described above demonstrates the use of ES cells according to the present invention in order to perform quantitative analysis of a regulatory sequence.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A preparation of non-inbred mouse embryonic stem (ES) cells that comprise alleles derived from at least three different inbred mouse strains, wherein the ES cells have good developmental potential and successfully compete with pre-existing inner cell mass cells, and their derivatives, when injected into a normal blastocyst.

2. The non-inbred ES cell preparation according to claim 1, wherein the ES cells additionally comprise a transgene docking site.

3. A preparation of non-inbred mouse embryonic stem (ES) cells that comprise alleles derived from at least two different inbred mouse strains and a transgene docking site, wherein the ES cells have good developmental potential and successfully compete with pre-existing inner cell mass cells, and their derivatives, when injected into a normal blastocyst.

4. The non-inbred ES cell preparation according to claim 2, wherein the transgene docking site is a deletion mutant of a hypoxanthine phosphoribosyltransferase (HPTR) gene.

5. The non-inbred ES cell preparation according to claim 2, wherein the transgene docking site comprises a loxP site.

6. The non-inbred ES cell preparation according to claim 1, wherein chimeras derived from the ES cells exhibit greater than 50% ES cell contribution.

7. The non-inbred ES cell preparation according to claim 6, wherein chimeras derived from the ES cells exhibit greater than 90% ES cell contribution.

8. The non-inbred ES cell preparation according to claim 7, wherein chimeras derived from the ES cells exhibit about 100% ES cell contribution.

9. A method for producing an ES cell-derived mouse comprising the steps of:
(a) introducing a non-inbred mouse ES cell preparation according to claim 1 into a normal mouse blastocysts or a tetraploid mouse blastocysts or aggregating a non-inbred mouse ES cell preparation according to claim 1 with one or more pre-implantation embryos under conditions that result in production of at least one embryo;
(b) transferring the resulting embryo(s) into an appropriate foster mother; and
(c) maintaining the foster mother under conditions that result in development of live offspring.

10. A method for producing an ES cell-derived, transgenic mouse comprising the steps of:
(a) introducing one or more transgenic sequences into non-inbred mouse ES cells of an ES cell preparation according to claim 2;
(b) maintaining the ES cells under conditions that result in homologous recombination at the transgene docking site such that the one or more transgenic sequences are incorporated in the genome of the ES cells;
(c) introducing the resultant recombinant ES cells into normal blastocysts(s) or tetraploid blastocysts(s) or said recombinant ES cells with one or more pre-implantation embryos, under conditions that result in production of at least one embryo;
(d) transferring the resulting embryo(s) into an appropriate foster mother; and
(e) maintaining the foster mother under conditions that result in development of live offspring, wherein the ES cells have good developmental potential.

11. A method for producing an ES cell-derived, gene targeted mouse comprising the steps of:
(a) performing a genetic alteration or mutation of one or more genes or parts of genes in non-inbred mouse ES cells of an ES cell preparation according to claim 1;
(b) maintaining the ES cells under conditions that result in homologous recombination such that the knock-out is incorporated in the genome of the ES cells;
(c) introducing the resultant recombinant ES cells into normal blastocyst(s) or tetraploid blastocysts(s) or said recombinant ES cells with one or more pre-implantation embryos, under conditions that result in production of at least one embryo;
(d) transferring the resulting embryo(s) into an appropriate foster mother; and
(e) maintaining the foster mother under conditions that result in development of live offspring, wherein the ES cells have good developmental potential.

12. The method according to claim 9, wherein the appropriate foster mother is, a pseudopregnant female mouse.

13. An ES cell-derived mouse that is prepared according to the method of claim 9.

14. The mouse according to claim 13, which is a transgene bearing mouse.

15. The mouse according to claim 13, which is a genetically altered or mutated mouse.

16. A method for preparing mouse embryonic stem cells having good developmental potential that comprises the steps of:
(a) mating a female mouse of a first inbred mouse strain with a male mouse of a second inbred mouse strain, wherein the first and the second mouse strains are different;
(b) performing multiple generations of breeding including a combination of at least one cross and at least one backcross from offspring obtained from the mating between the female mouse and the male mouse in step (a);
(c) recovering blastocysts from a mouse obtained following the multiple generations of breeding performed in step (b); and
(d) deriving embryonic stem cells from the inner cell masses of said blastocysts.

17. A method for preparing mouse embryonic stem cells having good developmental potential that comprised the steps of:
(a) mating a female mouse of a first inbred mouse strain with a male mouse of a second inbred mouse strain, wherein the first and the second mouse strains are different;
(b) mating an offspring of the mating of step (a) or an offspring of a subsequent generation with a mouse of a third inbred mouse strain;
(c) performing multiple generations of breeding including a combination of a least one cross and at least one backcross from offspring obtained from the mating of step (b);
(d) recovering blastocysts from a mouse obtained following the multiple generations of breeding performed in step (c) and
(e) deriving embryonic stem cells from the inner cell masses of said blastocysts.

18. The method according to claim 16, wherein the multiple generations of breeding comprises a combination of 5 or 6 crosses and backcrosses.

19. The method according to claim 16, wherein at least one of the inbred mouse strains contains a transgene docking site.

20. The method according to claim 19, wherein the transgene docking site is a deletion mutant of a hypoxanthine phosphoribosyltransferase (HPTR) gene.

21. The method according to claim 19, wherein the transgene docking site comprises a loxP site.

22. A non-inbred embryonic stem (ES) cell preparation obtained by the method of claim 16.

23. Use of the ES cell preparation according to claim 1 for producing an ES cell derived mouse.

24. Use of the ES cell preparation according to claim 1 for producing an ES cell derived genetically modified mouse.

25. The use according to claim 24, wherein said genetically modified mouse is a transgenic mouse.

26. The use according to claim 24, wherein said genetically modified mouse comprises a genetic alternation or mutation.
